(19) United States
(12) Patent Application Publication
(10) Pub. No.: US 2018/0256054 A1
(43) Pub. Date: Sep. 13, 2018

(54) SYSTEM FOR MONITORING AND EVALUATING CARDIAC ANOMALIES

(71) Applicant: NATIONAL CARDIAC, INC., Escondido, CA (US)

(72) Inventors: Paul M. DiPerna, Escondido, CA (US); Freeman H. Rose, JR., Del Mar, CA (US)

(21) Appl. No.: 15/454,459

(22) Filed: Mar. 9, 2017

(51) Int. Cl. A61B 5/04; A61B 5/00; A61B 5/0402; A61B 5/11

(52) U.S. Cl. CPC A61B 5/04012; A61B 5/6823; A61B 5/0402; A61B 5/6867; A61B 5/11; A61B 5/7271; A61B 5/746

(57) ABSTRACT

A system for monitoring and evaluating cardiac anomalies of a patient requires a cardiac sensor for detecting cardiac signals, and a perturbation sensor for detecting perturbations caused by stimuli that influence the cardiac signals. Based on normal cardiac signals for the patient, a cardio-profile is created which includes ranges of acceptable variations in selected parameters for the waveform of the cardiac signals. When a perturbation is detected by the perturbation sensor, its influence on the corresponding cardiac signal is evaluated relative to the cardio-profile to determine whether an anomaly has occurred. If so, the perturbation and the cardiac signal are further evaluated to determine whether a medical response is required.

### FIELD OF THE INVENTION

The present invention pertains generally to systems for monitoring and evaluating cardiac anomalies. In particular, the present invention pertains to systems for monitoring and evaluating cardiac signals relative to a predetermined cardio-profile, to thereby identify anomalies in the cardiac signal. The present invention is particularly, but not exclusively useful as a system that evaluates perturbations which cause cardiac anomalies for the purpose of determining whether appropriate medical action is required.

### BACKGROUND OF THE INVENTION

An electrocardiogram (EKG) is essentially a record of the electrical currents that are associated with a patient's heart muscle activity. More specifically, the EKG record is represented by a curve which depicts a sequence of waveforms, wherein each waveform is representative of heart muscle activity during a corresponding heart function cycle. In a normal EKG, the waveforms will comply with well-known norms. In detail, each waveform is characterized by measurable (observable) parameters which include: waveform shape, amplitudes within the waveform, the repetition rate of the waveforms in the EKG, variability of the repetition rate, and waveform discontinuities. Deviations from the norm are therefore observable as abnormalities (i.e. anomalies) which may require further scrutiny.

The source or cause of a cardiac anomaly may not always be easily determined. For instance, cardiac anomalies can be caused by many factors and can result from either environmental or physical considerations. Moreover, these factors may be either external or internal factors. In each instance, however, the occurrence of a significant anomaly can typically be detected as a perturbation in a cardiac signal's waveform.

In the event, it can sometimes happen that a cardiac anomaly does not require medical attention. For example, it sometimes happens that a person (patient) is influenced by an external or internal stimulus that, although it may be temporarily disruptive, it may not be detrimental to the person's overall health or well-being. In such cases, the anomaly can be ignored. Obviously, an ability to determine the difference between anomalies that require medical treatment, and those that do not, is an important health consideration.

With the above in mind, it is an object of the present invention to provide a cardio-profile which can be used as a reference by a monitoring system to determine when a cardiac anomaly occurs. Another object of the present invention is to provide a monitoring system that identifies when cardiac anomalies are caused by perturbations that result from identifiable environmental and/or physical conditions. Still another object of the present invention is to evaluate perturbations which cause a cardiac anomaly and thereby determine whether the perturbation justifies an appropriate medical response. Yet another object of the present invention is to provide a system for monitoring and evaluating cardiac anomalies that is relatively simple to manufacture, is easy to use, and is comparatively cost effective.

### SUMMARY OF THE INVENTION

In accordance with the present invention, a system is provided for monitoring and evaluating cardiac anomalies. In particular, the present invention detects each anomaly by electromagnetically monitoring the heart muscle of a patient for perturbations to its waveform(s) that may occur during a heart muscle cycle(s). However, recognizing that not all perturbations cause cardiac anomalies which require medical attention, the present invention evaluates only anomalies that do not comply with a predetermined, patient-specific, cardio-profile. Specifically, further evaluation of a non-compliant anomaly, together with an evaluation of the nature of the perturbation that caused the anomaly, is done to determine whether medical attention is required.

Structurally, the system of the present invention includes a cardiac sensor and an array or perturbation sensors. In this combination, the cardiac sensor (e.g, an electrocardiograph) is used for detecting cardiac signals (i.e. EKG waveforms), and the array of perturbation sensors is used for sensing different stimuli that can influence a patient's health and well-being. Specifically, the perturbation sensors are selected to sense stimuli that will cause a perturbation to the EKG waveform. Importantly, both the cardiac sensor and the array of perturbation sensors are separately connected to a same signal processor which is a component of a computer.

For one embodiment of the present invention, the sensors and the other computer components can be mounted directly onto the body of a patient (e.g. onto the torso of the patient). In an alternate embodiment of the present invention, the signal processor and other computer components may be located at a remote site. In this latter case, a transceiver remains located with the sensors on the patient's body where it is used for wireless communication with the signal processor which is located at the remote site (facility).

With specific reference to the array of perturbation sensors, they can be individually selected to sense/detect respectively different stimuli that are context relevant for a diagnosis. For example, some sensors will be selected to sense changes in environmental conditions that result in perturbations of a cardiac signal such as: weather conditions, electromagnetic conditions, radioactivity, time of day, climatic considerations, and altitude. Other perturbation sensors will be selected to sense changes in physical conditions that result in perturbations of a cardiac signal such as: stress, trauma, disease, exercise/activity level, extrinsic activities, sleep patterns, and body contacts. Still other perturbation sensors will be selected to sense patient compliance with indications and instructions for proper use and maintenance of the system. Examples of various sensors which can be used for the above-noted perturbation factors include an accelerometer, a thermometer, a clock, a photoelectric cell, a chemical detector, a microphone, a Geiger counter, a camera, an electromagnetic wave detector, and a battery charge and system readiness detector.

As noted above, it is an important feature of the present invention that the signal processor will incorporate a cardio-profile. In detail, this cardio-profile establishes acceptable ranges for variations in selected parameters of characteristic cardiac signals. These selected parameters will typically include: a waveform shape, amplitudes within the waveform, the repetition rate of the waveform, and discontinuities in the waveform.

In addition to the cardio-profile and the signal processor, other computer components include a comparator and an evaluator. Specifically, the comparator is used to compare cardiac signals that are detected by the cardiac sensor with the cardio-profile. Based on this comparison, anomalies that are caused by perturbations, and which are non-compliant with the cardio-profile, are identified. The evaluator then evaluates the particular perturbation-caused-anomaly, together with the nature of the perturbation, to determine whether a response action (i.e. medical attention) is necessary.

For embodiments of the present invention wherein the signal processor and other computer components are mounted directly on the patient, a transceiver can be connected with the signal processor for transmitting a report to a remote facility whenever the evaluator determines an anomaly requires an active medical response. In such an embodiment, the transceiver can also be used for receiving information from the remote facility to update the cardio-profile when needed. As indicated above, for an alternate embodiment of the present invention, the signal processor and computer components may be located at the remote facility. In this latter case, the transceiver can still be mounted with the sensors on the patient and used to transmit cardiac signals and perturbation signals to the signal processor at the remote site.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of components for the system of the present invention shown as a self-contained unit which is wearable on a patient;

FIG. 2 is an alternate embodiment of the present invention showing signal processing and other computer components of the present invention, when they are separated from a patient and located at a remote facility;

FIG. 3 is a representative electrocardiogram of a patient showing characteristic measurable parameters of a heart muscle signal during respective heart muscle cycles;

FIG. 3A is a graph of a heart muscle signal during a single heart muscle cycle (i.e, a single heart beat), taken from the electrocardiogram shown in FIG. 3; and

FIG. 3B is a graph of sequential heart muscle cardiac signals showing an exemplary tolerable variation range, r, for use in a cardio-profile required for the present invention, and a non-compliant deviation in a subsequent cardiac signal which identifies a cardiac anomaly.

### DESCRIPTION OF THE PREFERRED EMBODIMENTS

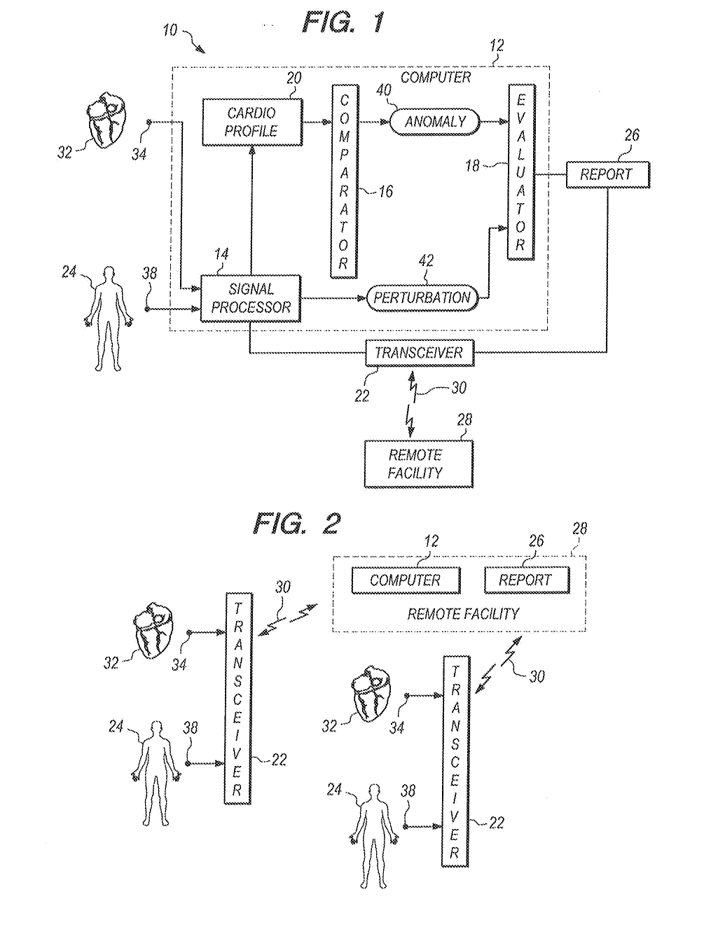

Referring initially to FIG. 1, a system in accordance with the present invention for monitoring cardiac irregularities (i.e. anomalies) is shown and is generally designated 10. As shown, the system 10 includes a computer 12, and within the computer 12 it incorporates a signal processor 14, a comparator 16 and an evaluator 18. Importantly, the computer 12 also includes a cardio-profile 20 that is disclosed below in detail.

In a preferred embodiment of the present invention, the computer 12 and a transceiver 22 are wearable on a user (patient) 24. For this embodiment, a report 26 which is generated by the computer 12 can be presented for direct viewing by the user (patient) 24. Additionally, the report 26 can be sent by the transceiver 22 to a remote facility 28 for review and consideration via a wireless connection 30.

For an alternate embodiment of the present invention as shown in FIG. 2, rather than being worn by the user (patient) 24, the computer 12 can be located at the remote facility 28. In this case, the transceiver 22 remains with, and is still wearable by, the user (patient) 24. For this alternate embodiment of the system 10, communication between the user (patient) 24 and the remote facility 28 will still be established via the wireless connection 30. As suggested by FIG. 2, a plurality of users (patients) 24 can use a same computer 12.

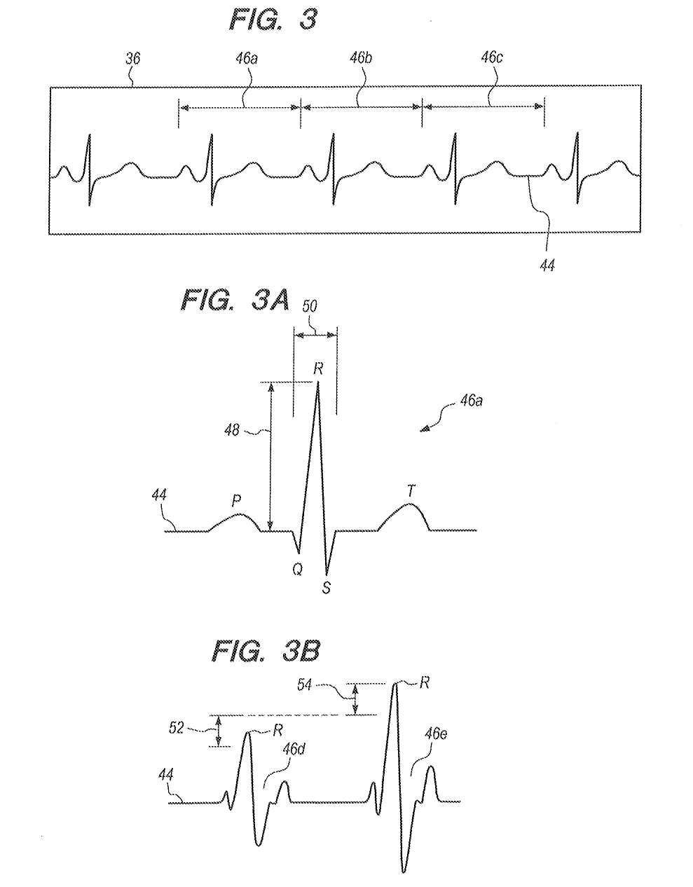

Returning to FIG. 1, it is to be appreciated that both the user (patient) 24 and the heart muscle 32 of the user (patient) 24 are connected by respective sensors with the computer 12. Specifically, with regard to the heart muscle 32, a cardiac sensor 34 is externally positioned on (attached to) the user (patient) 24 (i.e. extracorporeal). Specifically, this is done for the purpose of monitoring the activity of the heart muscle 32. Preferably the cardiac sensor 34 is an electrocardiograph of a type that is well known in the pertinent art for the purpose of generating an electrocardiogram (EKG) 36 (see FIG. 3). In accordance with the present invention, the EKG 36 is monitored to detect anomalies 40 in the activity of the heart muscle 32.

In addition to the cardiac sensor 34, a perturbation sensor 38, or an array including a plurality of perturbation sensors 38, is provided for the purpose of detecting and monitoring perturbations 42 that are caused by external influences and experienced by the user (patient) 24. As recognized by the present invention, these perturbations 42 can be physically or environmentally caused. For example, environmental perturbations can be caused by such factors as local weather conditions, electromagnetic radiations, radioactivity, time of day, climatic considerations, and altitude. On the other hand, physical perturbations 42 can be caused by such factors as stress, trauma, disease, extrinsic exercise/activity level, sleep patterns, and body contacts. Physical perturbations 42 can also include perturbations associated with patient compliance with instructions and indications for use as well as maintenance of the system. It is also recognized that each type of perturbation 42 may require a different type of perturbation sensor 38. With this in mind, examples of perturbation sensors 38 include: an accelerometer, a thermometer, a clock, a photoelectric cell, a chemical detector, a microphone, a Geiger counter, a camera, an electromagnetic wave detector and a battery charge and system readiness sensor. For example, a system sensor can be incorporated within the perturbation sensor 38 for monitoring an operational status of both the cardiac sensor 34 and the perturbation sensor 38. The overall purpose here is to detect system perturbations that can be respectively caused by patient compliance or non-compliance, as well as maintenance considerations. Typically, considerations for the operational status will include battery charge and operational readiness requirements.

Still referring to FIG. 1 it will be seen that the cardiac sensor 34 and the perturbation sensor 38 are both connected to the same signal processor 14. It is also seen that the signal processor 14 is connected to the cardio-profile 20 in the computer 12. With these connections, signals from the cardiac sensor 34 and from the perturbation sensor 38 will effectively arrive simultaneously at the signal processor 14. The signal processor 14 then separately distinguishes the cardiac signal from the perturbation signal and transfers the cardiac signal to the cardio-profile 20. The comparator 16 then evaluates the cardiac signal relative to the cardio-profile 20 and, as disclosed in detail below, determines whether a non-compliant anomaly 40 has occurred. Specifically, for purposes of the present invention, a non-compliant anomaly 40 results whenever a cardiac signal does not comply with the predetermined requirements of the cardio-profile 20.

FIG. 1 also shows that a non-compliant anomaly 40, and a simultaneously occurring perturbation 42, will be jointly evaluated at the evaluator 18. During this evaluation, the non-compliant anomaly 40 and the perturbation 42 are analyzed in context with each other for their relative severity, and for the nature of the influence the perturbation 42 has on the cardiac signal. The report 26 is the result of this evaluation.

The structure and functionality of the cardio-profile 20 will be best appreciated with reference to FIGS. 3, 3A and 3B. With reference to FIG. 3, the electrocardiogram (EKG) 36 is shown as a time record of a waveform 44 that represents activity of a heart muscle 32. As shown, the waveform 44 is clinically recognized as a continuing sequence of heart muscle cycles 46. It is well known that the time duration of each heart muscle cycle 46 can vary (i.e. exhibit different pulse rates as well as variations in those rates), and that the shape of the waveform 44 can also vary from one heart muscle cycle 46 to another. With this in mind, the waveform 44 and the heart muscle cycles 46a-c shown in FIG. 3 are considered exemplary of normal heart muscle 32 activity.

With reference to FIG. 3A, the heart muscle cycle 46a shows that the waveform 44 within each individual heart muscle cycle 46 is characterized by several identifiable features. In FIG. 3A, the departures P, Q, R, S and T of waveform 44 from a common base line are indicative of such features. As mentioned above, the waveform 44 within different heart muscle cycles 46 can have different shapes, and they can each be defined by different measurable parameters. For example, R of the QRS complex of waveform 44 of heart muscle cycle 46a has an amplitude 48, and the QRS complex itself has a time duration 50. Importantly, changes in the parameters of amplitude 48 and time duration 50 can be separately measured for each of the consecutive heart muscle cycles 46a-c. In this context, it is to be emphasized and appreciated that the parameters of amplitude 46 and time duration 50 that have been selected for this example are only representative of many other similar type parameters that can be selected for use from the same heart muscle cycles 46a-c.

With the above in mind, the present invention is provided to determine when anomalies 40 in a waveform 44 indicate that medical attention is required. In particular, the present invention is provided to detect anomalies 40, and to evaluate these anomalies 40 in context with a simultaneous perturbation 42. Further to the above disclosure, a cardio-profile 20 is provided in the computer 12. As implied, for the present invention the cardio-profile 20 is to be used as a reference for identifying anomalies 40 in the waveform 44 that require further evaluation.

In detail, the cardio-profile 20 is predetermined, and it is used as a so-called benchmark for the waveform 44. Stated differently, the cardio-profile 20 establishes what constitutes an acceptable waveform 44, and it thus identifies a non-compliant waveform 44 as an anomaly 40. Detailed disclosure for using the cardio-profile 20 to identify an anomaly (i.e. a non-compliant waveform 44) is provided with reference to FIG. 3B.

In FIG. 3B, consecutive pulses of heart muscle 32 are shown for exemplary heart muscle cycles 46d and 46e. Recall, the waveform 44 in each of the cycles 46d and 46e can have several different measureable parameters, and each can differ sequentially from one cycle 46 to the next. With this in mind, consider R of the waveform 44 by itself. In this case, R in the heart muscle cycle 46d is considered normal for the particular user (patient) 24. Accordingly, a range 52 is established for the parameter R in the cardio-profile 20. As such, variations in R within the range 52 would comply with the cardio-profile 20. On the other hand, R in the heart muscle cycle 46e exhibits a deviation 54 in amplitude which is beyond the range 52. It would therefore be non-compliant with the cardio-profile 20. In this case, R in the heart muscle cycle 46e is evidence of a non-compliant waveform 44, and it would be identified as an anomaly 40 by the computer 12. In turn, it would be subject to further evaluation with a simultaneous perturbation 42, and possible consideration for medical attention in report 26. It is to be appreciated that medical attention could include attention by a physician, or attention delegated by the physician, to include, for example, instructions education and reminders pertaining to patient compliance, system maintenance and upkeep.

While the particular System for Monitoring and Evaluating Cardiac Anomalies as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for monitoring and evaluating cardiac anomalies which comprises:
a cardiac sensor positioned in contact with the torso of a patient for detecting signals generated by the heart muscle of the patient, wherein the signals are characterized by measurable parameters;
a perturbation sensor positioned on the patient for detecting environmental and physical perturbations, wherein a perturbation influences at least one parameter of the signals detected by the cardiac sensor;
a signal processor connected to the cardiac sensor and to the perturbation sensor, wherein the signal processor includes a predetermined cardio-profile, wherein the cardio-profile is patient-specific and establishes acceptable ranges for variations in the individual parameters of the signals detected by the cardiac sensor;
a comparator incorporated into the signal processor for comparing a cardiac signal detected by the cardiac sensor with the cardio-profile to identify a non-compliant anomaly; and
an evaluator incorporated into the signal processor for evaluating the non-compliant anomaly with the nature of the perturbation to determine whether an appropriate medical response is required.

2. The system recited in claim 1 wherein the measurable parameters of signals from the heart muscle are selected from the group consisting of a waveform shape, amplitudes within the waveform, repetition rate of the waveform, variability of the repetition rate, and discontinuities in the waveform and wherein an anomaly is identified when a perturbation extends beyond an acceptable range in the cardio-profile.

3. The system recited in claim 1 wherein components selected for the perturbation sensor are selected from the group consisting of an accelerometer, a thermometer, a clock, a photoelectric cell, a chemical detector, a microphone, a Geiger counter, a camera, and an electromagnetic wave detector.

4. The system recited in claim 1 wherein the environmental perturbations are respectively caused by local weather conditions, electromagnetic radiations, radioactivity, time of day, climatic considerations, and altitude.

5. The system recited in claim 1 wherein the physical perturbations are respectively caused by stress, trauma, disease, extrinsic exercise/activity level, sleep patterns, and body contacts.

6. The system recited in claim 1 further comprising a system sensor incorporated with the perturbation sensor for monitoring an operational status of the cardiac sensor and the perturbation sensor, to detect system perturbations respectively caused by patient compliance and system maintenance considerations, to include battery charge and operational readiness requirements.

7. The system recited in claim 1 further comprising a transceiver connected with the signal processor for transmitting an alert to a remote facility whenever the evaluator determines an anomaly requires an active medical response, and for receiving information from the remote facility to update the cardio-profile when needed.

8. The system recited in claim 1 wherein the cardiac sensor and the perturbation sensor are mounted on a same substrate.

9. The system recited in claim 1 wherein the cardiac sensor is implanted in the torso of the patient.

10. The system recited in claim 1 wherein the cardiac sensor is an electrocardiograph, and wherein the perturbation sensor is an array of sensors, wherein each sensor in the array detects a respectively different perturbation.

11. A system for monitoring and evaluating cardiac anomalies which comprises:
an electrocardiograph (EKG) for recording cardiac signals of a patient, wherein the cardiac signals are each characterized by measurable signals and are recorded sequentially as a plurality of heart function cycles, and wherein each heart function cycle has a unique waveform;
at least one perturbation sensor for detecting environmental and physical perturbations of the waveform in a cardiac signal during a heart function cycle;
a signal processor connected to the EKG and to the perturbation sensor, wherein the signal processor includes a predetermined cardio-profile, wherein the cardio-profile is patient-specific and establishes acceptable ranges for variations in the individual parameters of the signals detected by the cardiac sensor; and
a computer incorporated into the signal processor for comparing a cardiac signal detected by the EKG with the cardio-profile to identify an anomaly, and for evaluating the anomaly with the perturbation of the waveform to determine whether an appropriate response action is required.

12. The system recited in claim 11 wherein the measurable parameters of signals from the heart muscle are selected from the group consisting of a waveform shape, amplitudes within the waveform, the repetition rate of the waveform, variability of the repetition rate, and discontinuities in the waveform, and wherein an anomaly is identified when a perturbation extends beyond an acceptable range in the cardio-profile,

13. The system recited in claim 11 wherein components selected for the perturbation sensor are selected from the group consisting of an accelerometer, a thermometer, a clock, a photoelectric cell, a chemical detector, a microphone, a Geiger counter, and an electromagnetic wave detector, wherein the environmental perturbations are respectively caused by local weather conditions, electromagnetic radiations, radioactivity, time of day, climatic considerations and altitude, and wherein the physical perturbations are respectively caused by stress, disease, extrinsic activity, sleep patterns, and body contacts.

14. The system recited in claim 11 further comprising a system sensor incorporated with the perturbation sensor for monitoring an operational status of the EKG and the perturbation sensor, to detect system perturbations respectively caused by patient compliance and system maintenance considerations, to include battery charge and operational readiness requirements.

15. The system recited in claim 11 further comprising a transceiver connected with the signal processor for transmitting an alert to a remote facility whenever the evaluator determines an anomaly requires an active medical response, and for receiving information from the remote facility to update the cardio-profile when needed.

16. The system recited in claim 11 wherein the cardiac sensor and the perturbation sensor are mounted on a same substrate.

17. A non-transitory, computer-readable medium having executable instructions stored thereon that direct a computer system to perform a process for monitoring and evaluating cardiac anomalies, the medium comprising instructions for:
establishing a cardio-profile for a patient, wherein the cardio-profile is patient-specific and identifies certain measurable parameters of cardiac signals generated by the heart muscle of a patient, and wherein the cardio-profile establishes acceptable ranges for variations in individual parameters of the signal;
detecting the cardiac signals generated by the heart muscle of the patient;
detecting perturbation signals experienced by the patient, wherein the cardiac signals and the perturbation signals are detected simultaneously;
comparing the cardiac signals with the cardio-profile to identify when a perturbation signal causes an anomaly; and
evaluating the perturbation signal to determine whether the resultant anomaly requires an appropriate action.

18. The medium recited in claim 17 wherein the measurable parameters of signals from the heart muscle are selected from the group consisting of a waveform shape, amplitudes within the waveform, the repetition rate of the waveform, and discontinuities in the waveform.

19. The medium recited in claim 17 wherein the perturbation includes environmental and physical perturbations, wherein the perturbation influences at least one parameter of the signals detected by the cardiac sensor, wherein the environmental perturbations are respectively caused by local weather conditions, electromagnetic radiations, radioactivity, time of day, climatic considerations and altitude, and wherein the physical perturbations are respectively caused by stress, disease, extrinsic activity, sleep patterns, and body contacts.

20. The medium recited in claim 17 further comprising instructions for:
transmitting an alert to a remote facility whenever an anomaly requires an active medical response; and
receiving information from the remote facility to update the cardio-profile when needed.

21. The method recited in claim 17 further comprising the steps of:
monitoring the steps detecting cardiac signals and detecting perturbation signals to determine an operational status of the computer system; and
transmitting an alert to a remote facility when the operational status of the computer system requires attention.
